(19) United States
(12) Patent Application Publication
(10) Pub. No.: US 2021/0393443 A1
(43) Pub. Date: Dec. 23, 2021

(54) ADAPTABLE ADHESIVE WOUND DRESSINGS

(71) Applicant: CONVATEC TECHNOLOGIES INC., Las Vegas, NV (US)

(72) Inventors: Jade Steven, Las Vegas, NV (US); Sophie Ballamy, Las Vegas, NV (US); Claire Brewer, Las Vegas, NV (US); Manjunath Penagondla, Las Vegas, NV (US)

(21) Appl. No.: 17/273,934

(22) PCT Filed: Sep. 6, 2019
(86) PCT No.: PCT/US2019/050086

§ 371 Date: Mar. 5, 2021

Related U.S. Application Data
(60) Provisional application No. 62/728,036, filed on Sep. 6, 2018.

(51) Int. Cl. A61F 13/02; A61L 15/28; A61L 15/26; A61L 15/42

(52) U.S. Cl. CPC A61F 13/0263; A61F 13/022; A61L 15/425; A61L 15/26; A61L 15/28

(57) ABSTRACT

Adaptable adhesive wound dressings are provided for wound management and promoting the healing of wounds.

### CROSS-REFERENCE TO RELATED APPLICATIONS

This application claims the benefit of U.S. Provisional Application No. 62/728,036 filed on Sep. 6, 2018. Priority is claimed pursuant to 35 U.S.C. § 119. The above noted patent application is incorporated by reference as if set forth fully herein.

### FIELD OF THE INVENTION

Described herein are adaptable adhesive wound dressings suitable to be adapted to provide optimal healing conditions for various types of wounds.

### BACKGROUND OF THE INVENTION

Wound dressings promote the healing of dermal injuries. Wound dressings can be used for the management of wounds and for protection of the skin, and to assist with preventing the development of pressure ulcers. Wound dressings are generally suitable for treating a variety of wounds, including chronic and acute wound types, such as infected wounds, venous ulcers, diabetic ulcers, burns, pressure ulcers, and surgical wounds. Wound healing is a complex dynamic process that results in the restoration of anatomic continuity and function. Wounds may be characterized by one of four stages during the healing process: the exudative phase, the proliferation phase, the reparative phase, or the epithelial maturation stage. An ideally healed wound includes wounds in which the dermis has returned to normal anatomic structure, function, and appearance.

Wounds can present in multiple forms. Wounds can range from heavily exuding to non-exuding throughout the healing process. As such, the ideal properties of the dressing interface may change depending upon the clinical use. For example, a highly exuding wound in some cases may benefit from a hydrophilic interface to manage exudate, whilst a low- to non-exuding wound in some cases may benefit from a more hydrophobic interface (e.g. silicone adhesive), to help maintain a moist wound environment. As such clinicians often desire a range of dressings to meet their clinical needs.

### SUMMARY OF THE INVENTION

Provided herein, in some embodiments, are wound dressings comprising a backing, a first layer (e.g., an absorbent layer, a porous layer, a foam layer, or combinations thereof), a second layer (e.g., a hydrophobic layer, a hydrophilic layer, an amphiphilic layer, any of which may or may not be perforated or fenestrated or may or may not contain apertures), and a release liner. In some embodiments, the wound dressings disclosed herein comprise a wound contact layer, wherein the wound contact layer is a hydrocolloid which can be hydrophilic and/or at least somewhat hydrophobic. In some embodiments, the wound dressings disclosed herein comprise at least a first part opposing or generally opposing a second part. In some embodiments, the wound dressings disclosed herein comprise a facilitator for removal of the release liner such as one or more tabs, threads, stitches, indentations, bidirectional releaser and combinations thereof. In some embodiments, the wound dressings disclosed herein comprise a release liner comprising at least a first part generally opposing or generally opposing a second part configured such that removal of the release liner from the first part to the second part exposes at least a portion of the first layer, and removal of the release liner from the second part to the first part exposes at least a portion of the perforated hydrophobic layer. In some embodiments, disclosed herein are wound dressings comprising a backing, a first layer, an adherent layer, and a release liner comprising at least a first part located opposing or generally opposing a second part configured such that removal of the release liner from the first part to the second part exposes the first layer, and removal of the release liner from the second part to the first part exposes the adherent layer. In some embodiments, the wound dressings disclosed herein comprise a release liner configured such that removal of the release liner from the first edge to the second edge exposes the first layer by dissociating at least a part of the second layer from the wound dressing and at least a part of the second layer remains associated with the release liner. In some embodiments, the second layer is an adherent layer. In some embodiments, the wound dressings disclosed herein are configured such that dissociating the second layer comprises creating a window into the second layer through which the first layer may contact a wound. In some embodiments, the window is substantially unobstructed. In some embodiments, the wound dressings disclosed herein are configured such that dissociating the second layer comprises creating a substantially unobstructed window into the second layer through which the first layer may contact a wound.

In some embodiments, disclosed herein are wound dressings comprising a backing, an absorbent layer, a perforated hydrophobic layer, and a release liner. In some embodiments, the wound dressings disclosed herein comprise at least a first edge located opposite to a second edge. In some embodiments, the wound dressing disclosed herein comprise at least a first edge located parallel to a second edge. In some embodiments, the wound dressings disclosed herein comprise at least a first edge located perpendicular to a second edge. In some embodiments, the wound dressings disclosed herein comprise a method for removal of the release liner selected from the group consisting of tabs, threads, stitches, indentations, and bidirectional release. In some embodiments, the wound dressings disclosed herein comprise a release liner comprising at least a first edge located opposite to a second edge configured such that removal of the release liner from the first edge to the second edge exposes the absorbent layer, and removal of the release liner from the second edge to the first edge exposes the perforated hydrophobic layer. In some embodiments, disclosed herein are wound dressings comprising a backing, an absorbent layer, an adherent layer, and a release liner comprising at least a first edge located opposite to a second edge configured such that removal of the release liner from the first edge to the second edge exposes the absorbent layer, and removal of the release liner from the second edge to the first edge exposes the adherent layer. In some embodiments, the wound dressings disclosed herein comprise a release liner configured such that removal of the release liner from the first edge to the second edge exposes the absorbent layer by dissociating the perforated hydrophobic layer from the wound dressing and the perforated hydrophobic layer remains associated with the release liner. In some embodiments, the perforated hydrophobic layer is an adherent layer. In some embodiments, the wound dressings disclosed herein are configured such that dissociating the perforated hydrophobic layer comprises creating a window into the perforated hydrophobic layer through which the absorbent layer may contact a wound. In some embodiments, the window is substantially unobstructed. In some embodiments, the wound dressings disclosed herein are configured such that dissociating the adherent layer comprises creating a substantially unobstructed window into the adherent layer through which the absorbent layer may contact a wound.

In some embodiments, provided herein are wound dressing comprising a backing, an absorbent layer, a perforated hydrophobic layer, and a release liner comprising at least a first edge located opposite to a second edge, where the release liner is configured such that removal of the release liner from the second edge to the first edge exposes the perforated hydrophobic layer by dissociating the release liner from the wound dressing and the perforated hydrophobic layer remains associated with the wound dressing. In some embodiments, the wound dressings disclosed herein comprise an absorbent layer selected from the group consisting of gelling fiber, absorbent fiber, hydrophilic foam, antimicrobial fibers, superabsorbent powder-fiber blends, non-gelling fibers, and combinations thereof. In some embodiments, the absorbent layer is a hydrophilic or non-adherent layer. In some embodiments, the wound dressings disclosed herein comprise a non-adherent layer selected from the group consisting of gelling fiber, absorbent fiber, hydrophilic foam, antimicrobial fibers, superabsorbent powder-fiber blends, non-gelling fibers, and combinations thereof. In some embodiments, the wound dressings disclosed herein comprise an absorbent layer comprising gel forming fibers bound to a foam layer. In some embodiments, the wound dressings disclosed herein comprise a non-adherent layer comprising gel forming fibers bound to a foam layer. In some embodiments, the wound dressings disclosed herein comprise an absorbent layer comprising gel forming fibers bound to a foam layer comprising hydrophilic foam. In some embodiments, the wound dressings disclosed herein comprise an absorbent layer comprising gel forming fibers that is not bound to a foam layer comprising hydrophilic foam. In some embodiments, the wound dressings disclosed herein comprise an absorbent layer comprising gel forming fibers bound to an additional layer between the absorbent layer comprising gel forming fibers and a foam layer comprising hydrophilic foam. In some embodiments, the foam layer comprises hydrophilic foam. In some embodiments, the wound dressings disclosed herein comprise layers bound to each other. In some embodiments, the wound dressings disclosed herein comprise layers that are not bound to each other. In some embodiments, the wound dressings disclosed herein comprises layers that are placed or laid on top of each other. In some embodiments, the wound dressings disclosed herein comprise a backing, an absorbent layer, a perforated hydrophobic layer, and a release liner, wherein the backing, the absorbent layer, the perforated hydrophobic layer, and the release liner are bound to each other. In some embodiments, the wound dressings disclosed herein comprise a backing, an absorbent layer, a perforated hydrophobic layer, and a release liner, wherein the backing, the absorbent layer, the perforated hydrophobic layer, and the release liner are not bound to each other. In some embodiments, the wound dressings disclosed herein comprise a backing, an absorbent layer, a perforated hydrophobic layer, and a release liner, wherein at least one of the group selected from the backing, the absorbent layer, the perforated hydrophobic layer, and the release liner are bound to at least one other of the group selected from an absorbent layer, a perforated hydrophobic layer, and a release liner.

In some embodiments, the wound dressings disclosed herein comprise a backing, an absorbent layer, a perforated hydrophobic layer, and a release liner, and the absorbent layer comprises a material selected from the group consisting of chemically modified cellulose, alginate, superabsorbent fibers, synthetic fibers, and combinations thereof. In some embodiments, the chemically modified cellulose of the wound dressings disclosed herein is carboxymethylcellulose or sodium carboxymethylcellulose. In some embodiments, the wound dressings disclosed herein comprise an adhesive layer overlying the absorbent layer and extending beyond a periphery of the absorbent layer so as to adhere the wound dressing to skin surrounding a wound. In some embodiments, the wound dressings disclosed herein comprise an adhesive layer is selected from the group consisting of silicone adhesive, hydrocolloid adhesive, polyurethane adhesive, rubber-based adhesive, hydrogel, acrylic adhesive and combinations thereof. In some embodiments, the wound dressings disclosed herein comprise a perforated hydrophobic layer selected from the group consisting of silicone adhesive, hydrocolloid adhesive, polyurethane adhesive, rubber-based adhesive, hydrogel, acrylic adhesive, coated woven material or film, non-woven material, knitted material, electrospun material, and combinations thereof. In some embodiments, the wound dressings disclosed herein comprise a backing comprising polyurethane film. In some embodiments, the wound dressings disclosed herein comprise a backing comprising polyurethane film laminated to polyurethane foam. In some embodiments, the wound dressings disclosed herein comprise a backing comprising polyurethane film laminated to a fibrous component. In some embodiments, the fibrous component is selected from the group consisting of gelling fiber, absorbent fiber, hydrophilic foam, antimicrobial fibers, superabsorbent powder-fiber blends, non-gelling fibers, and combinations thereof. In some embodiments, the fibrous component is selected from the group consisting of gelling fiber, absorbent fiber, antimicrobial fibers, superabsorbent powder-fiber blends, non-gelling fibers, and combinations thereof.

In some embodiments, disclosed herein are wound dressings comprising a backing, an adhesive layer, an absorbent layer, and a perforated hydrophobic layer encapsulating said absorbent layer on all sides except for a first side, and the perforated hydrophobic layer is configured such that either the first side or a second side opposite to the first side may be adhered to the adhesive layer. In some embodiments, the wound dressings disclosed herein comprise a perforated hydrophobic layer encapsulating the absorbent layer creating a window through which absorbent layer may contact a wound. In some embodiments, the window is a substantially unobstructed window and provides an exposed region of the perforated hydrophobic layer for application to a wound. In some embodiments, the wound dressings disclosed herein comprise an absorbent layer selected from the group consisting of gelling fiber, non-gelling fiber, absorbent fiber, and hydrophilic foam, or combinations thereof. In some embodiments, the wound dressings disclosed herein comprise an absorbent layer comprising gel forming fibers bound to a foam layer. In some embodiments, the wound dressings disclosed herein comprise an absorbent layer comprising gel forming fibers that is not bound to a foam layer comprising hydrophilic foam. In some embodiments, the wound dressings disclosed herein comprise an absorbent layer comprising gel forming fibers bound to an additional layer between the absorbent layer comprising gel forming fibers and a foam layer comprising hydrophilic foam. In some embodiments, the wound dressings disclosed herein comprise a foam layer comprising hydrophilic foam. In some embodiments, the wound dressings disclosed herein comprise a backing, an adhesive layer, an absorbent layer, and a perforated hydrophobic layer encapsulating the absorbent layer on all but a first side and the perforated hydrophobic layer is configured such that either the first side or a second side opposite to the first side may be adhered to the adhesive layer, and the absorbent layer comprises a material selected from the group consisting of chemically modified cellulose, alginate, superabsorbent fibers, synthetic fibers, and combinations thereof. In some embodiments, the chemically modified cellulose is carboxymethylcellulose or sodium carboxymethylcellulose.

In some embodiments, the wound dressings disclosed herein comprise a backing, an adhesive layer, an absorbent layer, and a perforated hydrophobic layer encapsulating the absorbent layer on all but a first side and the perforated hydrophobic layer is configured such that either the first side or a second side opposite to the first side may be adhered to the adhesive layer, and the adhesive layer comprises a layer of foam situated between two layers of sodium carboxymethylcellulose. In some embodiments, the adhesive layer is selected from the group consisting of silicone adhesive, hydrocolloid adhesive, polyurethane adhesive, rubber-based adhesive, hydrogel, and acrylic adhesive, and combinations thereof. In some embodiments, the wound dressings disclosed herein comprise a perforated hydrophobic layer selected from the group consisting of silicone adhesive, hydrocolloid adhesive, polyurethane adhesive, rubber-based adhesive, acrylic adhesive, non-woven material, knitted material, and coated woven material. In some embodiments, the wound dressings disclosed herein comprise a backing comprising polyurethane film. In some embodiments, the wound dressings disclosed herein comprise a backing comprising polyurethane film laminated to polyurethane foam. In some embodiments, the wound dressings disclosed herein comprise a backing comprising polyurethane film laminated to a fibrous component. In some embodiments, the fibrous component is selected from the group consisting of gelling fiber, absorbent fiber, hydrophilic foam, antimicrobial fibers, superabsorbent powder-fiber blends, non-gelling fibers, and combinations thereof. In some embodiments, the wound dressings disclosed herein comprise a backing, an adhesive layer, and absorbent layer, and a perforated hydrophobic layer, wherein the adhesive layer and the perforated layer are made of the same material. In some embodiments, the wound dressings disclosed herein comprise a backing, an adhesive layer, and absorbent layer, and a perforated hydrophobic layer, wherein the adhesive layer and the perforated layer are both made of a material selected from the group consisting of silicone adhesive, hydrocolloid adhesive, polyurethane adhesive, rubber-based adhesive, hydrogel, non-woven material, knitted material, coated woven material, acrylic adhesive, and combinations thereof. In some embodiments, the wound dressings disclosed herein comprise a backing, an adhesive layer, and absorbent layer, and a perforated hydrophobic layer, wherein the adhesive layer and the perforated layer are made of different material and do not permanently adhere to each other such that the absorbent layer can be removed from the adhesive layer without deforming the absorbent layer. In some embodiments, the wound dressings disclosed herein comprise a backing, an adhesive layer, and absorbent layer, and a perforated hydrophobic layer, wherein the adhesive layer and the perforated layer are made of different material and do not permanently adhere to each other such that the adhesive layer can be removed from the absorbent layer without deforming the absorbent layer.

In some embodiments, disclosed herein are methods of manufacturing a wound dressing comprising forming a backing, forming an absorbent layer, forming a perforated hydrophobic layer, and forming a release liner comprising at least a first edge located opposite to a second edge, wherein the release liner is formed such that removal of the release liner from the first edge to the second edge exposes the absorbent layer and removal of the release liner from the second edge to the first edge exposes the perforated hydrophobic layer. In some embodiments, disclosed herein are methods of manufacturing a wound dressing comprising forming a backing, forming an adhesive layer, forming an absorbent layer, and forming a perforated hydrophobic layer encapsulating said absorbent layer on all but a first side, wherein the perforated hydrophobic layer is formed such that either the first side or a second side opposite to the first side may be adhered to the adhesive layer.

In some embodiments, disclosed herein are methods of treating a wound comprising determining a level of exudate from said wound; and, applying a wound dressing comprising a backing, an absorbent layer, a perforated hydrophobic layer, and a release liner comprising at least a first edge located opposite to a second edge, configured such that removal of said release liner from said first edge to said second edge exposes said absorbent layer and removal of said release liner from said second edge to said first edge exposes said perforated hydrophobic layer, whereupon a determination that the wound is a medium to high-exuding wound, the next step comprises removal of the release liner from the first edge to the second edge exposing the absorbent layer by removal of the perforated hydrophobic layer simultaneous with removal of the release liner and application of the wound dressing such that the absorbent layer is in contact with said wound. In some embodiments, disclosed herein are methods of treating a wound comprising determining a level of exudate from said wound; and, applying a wound dressing comprising a backing, an absorbent layer, a perforated hydrophobic layer, and a release liner comprising at least a first edge located opposite to a second edge, configured such that removal of said release liner from said first edge to said second edge exposes said absorbent layer and removal of said release liner from said second edge to said first edge exposes said perforated hydrophobic layer, whereupon a determination that the wound is a low- to non-exuding wound, the next step comprises removal of the release liner from the second edge to the first edge exposing the perforated hydrophobic layer and application of the wound dressing such that the wound dressing protects the wound.

In some embodiments, the wound dressings disclosed herein provide at least two options: at least a first option where the wound dressing is suitable for medium to highly-exuding wounds, skin protection, and wound healing; and at least a second option where the wound dressing is suitable for low to non-exuding wounds and skin protection.

In some embodiments, the wound dressings disclosed herein comprise: (a) a backing; (b) an absorbent layer; (c) a perforated hydrophobic layer; and (d) a release liner comprising at least a first edge located opposite, parallel, or perpendicular to a second edge, wherein said release liner is configured such that removal of said release liner from said first edge to said second edge exposes said absorbent layer, and removal of said release liner from said second edge to said first edge exposes said perforated hydrophobic layer. In some embodiments, the wound dressings disclosed herein comprise: (a) a backing; (b) an absorbent layer; (c) a perforated hydrophobic layer; and (d) a release liner comprising at least a first edge located opposite to a second edge, wherein said release liner is configured such that removal of said release liner from said first edge to said second edge exposes said absorbent layer, and removal of said release liner from said second edge to said first edge exposes said perforated hydrophobic layer. In some embodiments, the wound dressings disclosed herein comprise: (a) a backing; (b) an absorbent layer; (c) a perforated hydrophobic layer; and (d) a release liner comprising at least a first edge located parallel to a second edge, wherein said release liner is configured such that removal of said release liner from said first edge to said second edge exposes said absorbent layer, and removal of said release liner from said second edge to said first edge exposes said perforated hydrophobic layer. In some embodiments, the wound dressings disclosed herein comprise: (a) a backing; (b) an absorbent layer; (c) a perforated hydrophobic layer; and (d) a release liner comprising at least a first edge located perpendicular to a second edge, wherein said release liner is configured such that removal of said release liner from said first edge to said second edge exposes said absorbent layer, and removal of said release liner from said second edge to said first edge exposes said perforated hydrophobic layer.

In some embodiments, the wound dressings disclosed herein comprise: (a) a backing; (b) an absorbent layer; (c) a perforated hydrophobic layer; and (d) a release liner comprising at least a first edge in a vertical plane and a second edge in a horizontal plane, wherein said release liner is configured such that removal of said release liner from said first edge to said second edge exposes said absorbent layer, and removal of said release liner from said second edge to said first edge exposes said perforated hydrophobic layer.

In one embodiment, the disclosed technology relates to a wound dressing comprising: (a) a backing; (b) an absorbent layer; (c) a perforated hydrophobic layer; and (d) a release liner comprising at least a first edge located opposite, parallel, or perpendicular to a second edge, or a first edge in a vertical plane and a second edge in a horizontal plane, wherein said release liner is configured such that removal of said release liner from said first edge to said second edge exposes said absorbent layer, and removal of said release liner from said second edge to said first edge exposes said perforated hydrophobic layer. In one embodiment, the disclosed technology relates to a wound dressing comprising: (a) a backing; (b) an absorbent layer; (c) a perforated hydrophobic layer; and (d) a release liner comprising at least a first edge located opposite to a second edge, or a first edge in a vertical plane and a second edge in a horizontal plane, wherein said release liner is configured such that removal of said release liner from said first edge to said second edge exposes said absorbent layer, and removal of said release liner from said second edge to said first edge exposes said perforated hydrophobic layer.

### BRIEF DESCRIPTION OF THE DRAWINGS

FIGS. 1A and 1B show embodiments of the multilayered wound dressings disclosed herein. FIG. 1A shows a multilayered wound dressing 101 with polyurethane (PU) film 102, PU foam 103, a HYDROFIBER® (ConvaTec, United Kingdom)/gelling fiber wound contact layer 104, and perforated silicone adhesive border 105. FIG. 1B shows a multilayered wound dressing with a cover layer (top) 106, layer 1 (which may comprise one or more layers) 107, and layer 2 108 (or wound contact layer) which may have openings such as perforations, apertures, fenestrations, or the like and which provides a window exposing layer 1 when at least part of the release liner is removed.

FIGS. 2A and 2B show embodiments of the multilayered wound dressing disclosed herein. FIG. 2A shows a multilayered wound dressing 201 with PU film 202, PU foam 203, a HYDROFIBER® (ConvaTec, United Kingdom)/gelling fiber wound contact layer 204, a perforated silicone adhesive 205. FIG. 2B shows a multilayered wound dressing with a cover layer 206 (top), layer 1 207 (which may comprise one or more layers), and layer 2 208 (or wound contact layer) with openings such as perforations, apertures, fenestrations, or the like.

FIGS. 3A and 3B show embodiments of the multilayered wound dressing disclosed herein. FIG. 3A shows a multilayered wound dressing 301 in which the wound interface is adapted through layer removal to provide either a hydrophilic absorbent wound interface or an open structure hydrophobic/hydrophilic wound interface. Removal of the hydrophobic layer exposing liner (left-hand side) by pulling from left to right 302 exposes the hydrophobic wound contact layer. Removal of the absorbent pad exposing liner by pulling from right to left 303 exposes the absorbent pad (and the zig-zag perforations 305 function to permit the hydrophobic layer to “unzip” in one direction only). FIG. 3B depicts an embodiment lacking the absorbent pad shown in FIG. 3A. Removal of the hydrophobic layer exposing liner (left-hand side) by pulling from left to right 306 exposes the hydrophobic wound contact layer. Removal of the absorbent pad exposing liner by pulling from right to left 307 exposes the wound contact layer (and the zig-zag perforations 308 function to permit the hydrophobic layer to “unzip” in one direction only).

FIGS. 4A and 4B show embodiments of the multilayered wound dressing disclosed herein. FIG. 4A shows a multilayered reversible wound dressing in which the wound interface is selected from either side of the wound dressing. One side of the wound dressing 401 is the absorbent pad 403 exposed with perforated silicone border. The other side of the wound dressing 402 is the perforated hydrophobic wound contact layer with obstructed absorbent pad. 404 is the perforated hydrophobic wound contact layer with obstructed absorbent pad without release liner. The multilayered wound dressing disclosed herein further comprises a backing film 405, perforated silicone adhesive layer 406, perforated silicone case 407 surrounding the absorbent pad 408 or 409. Absorbent pad 408 comprises absorbent pad with exposed wound contact layer and absorbent foam layer in the middle. Absorbent pad 409 comprises absorbent pad with perforated silicone wound contact layer with partial absorbent pad exposure. FIG. 4B shows a multilayered reversible wound dressing in which the wound interface is selected from either side of the wound dressing. One side of the wound dressing 410 is the absorbent pad 412 exposed with perforated silicone border. The other side of the wound dressing 411 is the perforated hydrophobic wound contact layer with obstructed absorbent pad. 413 is the perforated hydrophobic wound contact layer with obstructed absorbent pad without release liner. The multilayered wound dressing disclosed herein further comprises a backing film 414, perforated silicone adhesive layer 415, perforated silicone case 416 surrounding the absorbent pad 417 or 418. Absorbent pad 417 comprises absorbent pad with exposed wound contact layer and absorbent foam layer in the middle. Absorbent pad 418 comprises absorbent pad with perforated silicone wound contact layer with partial absorbent pad exposure

FIG. 5A shows a multilayered would dressing 501, wherein the wound dressing has an exposure window 502, an absorbent pad 503, and a series of discontinuities to allow for differential release of the hydrophobic layer or to let it remain in place.

FIG. 5B shows the multilayered wound dressing of FIG. 5A, wherein the hydrophilic layer 504 can be exposed by peeling the release liner 505 in a direction as indicated by arrow 506.

FIG. 5C shows the multilayered wound dressing of FIG. 5A, wherein the hydrophobic layer 507 of the multilayered wound dressing can remain intact by peeling the release liner 508 in a direction as indicated by arrow 509.

FIGS. 6A, 6B and 6C shows an embodiment of the multilayered wound dressing as disclosed herein. FIG. 6A shows a multilayered would dressing 601, wherein the wound dressing has an exposure window 602, an absorbent pad 603, and has a series of discontinuities to allow for differential release of the hydrophobic layer or to let it remain in place. FIG. 6B shows the multilayered wound dressing of FIG. 6A, wherein the hydrophilic layer 604 can be exposed by peeling the release liner 605 in a direction as indicated by arrow 606.

FIG. 6C shows the multilayered wound dressing of FIG. 6A, wherein the hydrophobic layer 607 of the multilayered wound dressing can remain intact by peeling the release liner 608 in a direction as indicated by arrow 609.

FIG. 7 shows schematic design of a multilayered wound dressing, where the location of the release liner relative to cuts made in the window determines removal or stay of the window when peeling the release liner. FIG. 7A shows the removal edge where the window has a larger surface area of adhesion between the cut 702 and the release liner 701. FIG. 7B shows a stay edge with release liner 703 that is positioned immediately below a cut line 704 of the window, reducing surface area of the window adhering to the release liner. When removing the release liner as indicated by the direction of the arrow in FIG. 7B, the window stays attached to the multilayered wound dressing as opposed to being peeled along with the release liner. Both FIG. 7A and FIG. 7B also illustrate the positions of the light (L) and tight (T) releases between the window and the release liner and between the window and the wound dressing.

FIG. 7C shows a view from a side of the release liner side of the multilayered wound dressing, illustrating the arrangement of L and T releases. On the left, a T release is positioned between the release liner 705 and the window 706. Additional adhesive 707 is added to create a tighter adhesion between the release liner 705 and the window 706. An example of additional adhesive 707 for the T release includes using a polyester (PET) tape. On the right side, an L release requires lighter adhesive. Therefore, there is no additional adhesive. An example of light adhesive for the L release includes low-density polyethylene (LDPE).

FIG. 7D shows a view from a polyurethane (PU) side of the multilayered wound dressing. On the left (the remove edge), an L release is used. On the right (stay edge), a T release is used. For example, a band of transfer tape is added to the right side for higher adhesion.

FIG. 8A shows the multilayered wound dressing where a release liner 801 is peeled from an edge in a direction as indicated by the arrows. Peeling the release liner in this particular direction removes a removable window 802 as indicated by the darkened rectangular shape with solid line. Removing the window also removes a hydrophobic layer, revealing a hydrophilic absorbent wound interface 803 underneath for use in treating a high exuding wound.

FIG. 8B shows the multilayered wound dressing where the release liner 804 as well as the removable window 805 (darkened square with solid lines) are fully peeled away. In such instance, the hydrophobic layer is removed to reveal the hydrophilic absorbent wound interface 806 underneath for use in treating a high exuding wound.

FIG. 9 shows a multilayered wound dressing where a release liner 901 is fully removed to reveal a removable window 902 (darkened square with solid lines) remaining intact on the wound dressing for use in treating a wound that is low or non-exuding. The hydrophobic layer 903 comprises both the window and non-window portions, will be applied to a wound with low or none exudate.

### DETAILED DESCRIPTION OF THE INVENTION

Wound dressings provide wound management purposes, including the protection of skin and the wound area, as well as the absorption of wound exudate, to assist in the prevention of, for example, development of pressure ulcers. Wounds can range from heavily exuding through to non-exuding throughout the healing process. Accordingly, wound dressing needs can also change depending upon the clinical presentation of the wound. For example, a highly exuding wound may require a hydrophilic interface for managing wound exudate, while a low to none exuding wound may benefit from a more hydrophobic interface to help maintain a moist wound environment. A solution to the use of a range of wound dressings would be a wound dressing in which the wound contact layer is adaptable by the clinician in use to fit the characteristics of the wound. There is a need for a multi-layered adaptable adhesive wound dressing that has a wound contact surface that can be adapted at the point of use.

Provided herein are wound dressings for wound management, wherein the wound dressing is adaptable to meet the needs of the wound. The wound dressings may comprise gel-forming polymers, non-gel-forming fibers, or a combination thereof. In some embodiments, the wound dressings described herein are used in methods to treat a wound in an animal. In further embodiments, the wound dressings described herein are used in methods to treat a chronic wound in an animal. In some embodiments, the wound dressings described herein are used in methods to treat a wound in a mammal. In further embodiments, the wound dressings described herein are used in methods to treat a chronic wound in a mammal.

### Wound Dressings

Disclosed herein, in certain embodiments, are wound dressings comprising a backing, a first layer and a second layer and a release liner comprising at least a first edge located opposite to a second edge. In some instances, the first layer, for example, is an absorbent layer, and the second layer is, for example, a perforated layer, for example a hydrophobic layer. In some embodiments, the release liner is configured so that the direction of removal of the release liner dictates whether the first layer, for example an absorbent layer, or the second layer, for example a perforated layer, is exposed for application to a wound. In some instances, the perforated layer is a hydrophilic or hydrophobic layer. In some embodiments, such as embodiments in which the dressings disclosed herein are placed on top of a separate layer, the release liner is configured so that the direction of removal of the release liner dictates whether the first layer, for example an absorbent layer, or the second layer, for example a perforated layer, is exposed for application to a hydrophilic layer. Also disclosed herein, in certain embodiments, are wound dressings comprising a backing, an absorbent layer, a perforated hydrophobic layer, and a release liner comprising at least a first edge opposite to a second edge. In some embodiments, the release liner is configured so that the direction of removal of the release liner dictates whether the absorbent layer or the perforated hydrophobic layer is exposed for application to a wound or hydrophilic layer. Further disclosed herein are wound dressings comprising a backing, an adhesive layer, an absorbent layer, and a perforated hydrophobic layer encapsulating the absorbent layer except for a window on a first side of the absorbent layer. In some embodiments, the wound dressings disclosed herein are reversible. In this configuration, the perforated hydrophobic layer is reversible so that either the window of exposed absorbent layer or the perforated hydrophobic layer is exposed for application to a wound. In some embodiments, the wound dressings described herein comprise a hydrophobic layer selected from the group consisting of a hydrophobic layer comprising perforations, a hydrophobic layer comprising cuts, a hydrophobic layer comprising slits, a hydrophobic layer comprising holes, a hydrophobic layer comprising apertures, a hydrophobic layer comprising discontinuities, and a hydrophobic layer comprising bevels.

Further disclosed herein are wound dressings comprising a backing, a first layer, an adhesive layer, and a second layer encapsulating the first layer except for a window on a first side of the first layer. In some instances, the first layer is an absorbent layer. In other instances, the second layer is a perforated layer. In yet other instances, the second layer is a perforated hydrophobic layer. In some embodiments, the wound dressings disclosed herein are reversible. In this configuration, the second layer is reversible so that either the window of exposed first layer or the second layer is exposed for application to a wound. In some embodiments, the wound dressings described herein comprise a second layer wherein the second layer is a hydrophobic layer selected from the group consisting of a hydrophobic layer comprising perforations, a hydrophobic layer comprising cuts, a hydrophobic layer comprising slits, a hydrophobic layer comprising holes, a hydrophobic layer comprising apertures, a hydrophobic layer comprising discontinuities, and a hydrophobic layer comprising bevels.

In some embodiments, the wound dressings disclosed herein comprise materials suitable for use in bandages. Suitable materials for bandages are non-irritating, durable, and flexible and include, by way of non-limiting examples, textiles of natural fiber (e.g., cotton, linen, and hemp), textiles of synthetic fiber (e.g., nylon, polyester, aramid, olefin, and acrylic), and plastic (e.g., polyvinyl chloride, low-density polyethylene, and polypropylene). In some embodiments, the wound dressings disclosed herein comprise wound dressing packaging and release liner systems. Suitable materials for wound dressing packaging and release liner systems are easily torn and include, by way of non-limiting examples, paper and waxed paper. In some embodiments, the wound dressings disclosed herein are dimensioned to fit different parts of the body, such as wound dressings dimensioned to fit a human finger, or a human knee. In some embodiments, the wound dressings disclosed herein are dimensioned in standard sizes, including for example, 5×5 cm, 6×7 cm, 10×12 cm, 10×20 cm, 15×20 cm, 30×20 cm, 40×20 cm, 50×20 cm, 20×20 cm, 25×25 cm, 30×30 cm, 35×35 cm, 40×40 cm, 45×45 cm, 50×50 cm, or sizes in-between any of these exemplary sizes. In some embodiments, the wound dressings disclosed herein are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 29, 40, 41, 42, 43, 44, 45, 46, 47, 48, 49, 50, or more millimeters long or wide, including increments therein. In some embodiments, the wound dressings disclosed herein are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 29, 40, 41, 42, 43, 44, 45, 46, 47, 48, 49, 50, or more centimeters long or wide, including increments therein. In some embodiments, suitable shapes for the wound dressings disclosed herein include square, rectangular, oval, round, and butterfly-shape wound dressings. In some embodiments, the wound dressings disclosed herein comprise wound dressings with multiple sites for the location of the absorbent layer and multiple sites for the location of the perforated hydrophobic layer.

### Backings

In some embodiments, the wound dressings described herein comprise a backing. In some embodiments, the backing is made from polyurethane (PU). In some embodiments, the backing is made from film or a non-woven material. In some embodiments, the backing is made from polyurethane film laminated to polyurethane foam or a non-woven material. In some embodiments, the polyurethane has a thickness between 0.02 mm and 0.04 mm. In some embodiments, the backing is transparent. In some embodiments, the backing has a high moisture vapor transmission rate (MVTR) and allows moisture to permeate through a wound dressing and evaporate from the wound. In some embodiments, the backing has a MVTR of at least 2,500 grams/meter²/day. In some embodiments, the backing has a MVTR in the range of from 1,000 grams/meter²/day to 30,000 grams/meter²/day, or from 9,000 grams/meter²/day to 27,000 grams/meter²/day. In some embodiments, the MVTR is measured using the liquid in contact ISO standard (see, e.g., British Standards Institution “Test methods for primary wound dressings; Moisture vapour transmission rate of permeable film dressings” BS EN 13726-2:2002 Section 3.3). In some embodiments, the backing is made from backing film or a strikethrough film. In some embodiments, the backing is printed to allow a user to indicate or mark the dressing, for example, with a marking device (such as a pen) which wound contact layer is exposed on the dressing to allow identification of the layer exposed without removal of the dressing.

### Absorbent Layers

In some embodiments, the wound dressings described herein comprise a first layer, wherein the first layer is an absorbent layer substantially insoluble in water. In some embodiments, the wound dressings described herein comprise an absorbent layer substantially insoluble in water. In some embodiments, the absorbent layer is a non-adherent layer. In some embodiments, the absorbent layer is an absorbent layer as described in WO2011/058311. In some embodiments, the absorbent layer comprises multiple layers. In some embodiments, the absorbent layer comprises foam. In some embodiments, the absorbent layer comprises fiber selected from the group consisting of sodium carboxymethylcellulose fiber, alginate fiber, chitosan or chitosan derivative fiber, acrylic fiber, non-gelling fiber, superabsorbent fiber, and combinations thereof. In some embodiments, the absorbent layer comprises an antimicrobial fiber, such as an antimicrobial fiber comprising silver ions or metal ions. In some embodiments, the wound dressings comprise one or more medicaments selected from the group consisting of an antibiotic, an anesthetic, an anti-inflammatory agent, a skin protective agent, and an odor-absorbing agent. In some embodiments, the fiber comprises chemically modified cellulose. In some embodiments, the fiber is carboxymethylcellulose fiber with a degree of substitution between 0.1 and 0.5 carboxymethyl groups per cellulose unit. In some embodiments, the carboxymethylcellulose fiber is a carboxymethylcellulose fiber as described in WO1993/012275. In some embodiments, the fiber is an acrylic fiber which incorporates a co-monomer and provides dye-sites in the fiber. In some embodiments, the co-monomer is selected from the group consisting of itaconic acid and 2-acrylamido methyl propane sulphonic acid. Where the fiber is an alginate fiber, it may be a calcium alginate fiber or a mixed metal alginate fiber such as a calcium/sodium alginate fiber. The alginate polymer may be one with a high mannuoronate or a high guluronate. In some embodiments, the wound dressings comprise an absorbent layer comprising chemically modified cellulose. In some embodiments, the wound dressings comprise an absorbent layer comprising, for example, carboxymethylcellulose, carboxyethylcellulose, or other chemically modified cellulose. In some embodiments, the carboxymethylcellulose is sodium carboxymethylcellulose. In some embodiments, the absorbent layer comprises HYDROFIBER® (ConvaTec, United Kingdom). In some embodiments, the wound dressings described herein provide advantages to the healing of the wound including the advantages of locking in exudate and trapping bacteria (including advantages such as protecting periwound skin and reducing maceration and minimizing wound and cross-infection during wound dressing removal), micro-contouring to a wound bed (including advantages such as minimizing “dead space” where bacteria can grow and maintaining the moisture balance in the wound bed), and responding to wound fluid levels forming a cohesive gel (including advantages such as forming a cohesive gel when the wound dressing comes in contact with exudates and providing a rapid and sustained antimicrobial activity on demand, such as when the wound dressing comprises ionic silver).

In some embodiments, the wound dressings described herein absorb exudate from a wound. In some embodiments, the wound dressings described herein comprise an absorbent layer with a minimum level of absorbency. In some embodiments, the absorbency of the wound dressings described herein may be measured by the free swell absorbency method. In some embodiments, the absorbency of the wound dressings described herein is at least 0.30 g/cm², or at least 0.40 g/cm², or at least 0.50 g/cm², or at least 0.60 g/cm², or at least 0.70 g/cm², or at least 0.80 g/cm², or at least 0.90 g/cm², or at least 1.0 g/cm², or at least 1.1 g/cm², or at least 1.2 g/cm², or at least 1.3 g/cm², or at least 1.5 g/cm², or at least 2.0 g/cm², or at least 2.5 g/cm². In some embodiments, the wound dressings described herein can achieve absorbency of at least 1.03 g/cm². In some embodiments, the wound dressings described herein comprise a gelling fiber, an absorbent fiber, or a hydrophilic foam. In some embodiments, the wound dressings disclosed herein comprise an absorbent core comprising a material selected from the group consisting of foam, polyurethane foam, absorbent textiles, hydrogels, superabsorbent fibers, superabsorbent powder-fiber blends, and mixtures thereof. In some embodiments, the absorbent core comprises a gelling blend of a material selected from the group consisting of foam, polyurethane foam, absorbent textiles, hydrogels, superabsorbent fibers, superabsorbent powder-fiber blends, and mixtures thereof. In some embodiments, the absorbent core comprises a non-gelling blend of a material selected from the group consisting of foam, polyurethane foam, absorbent textiles, hydrogels, superabsorbent fibers, superabsorbent powder-fiber blends, and mixtures thereof. In some embodiments, the wound dressings disclosed herein comprise an absorbent hydrophilic layer comprising a material selected from the group consisting of HYDROFIBER® (ConvaTec, United Kingdom), gelling fiber, gelling fiber blend, gelling fiber-synthetic fiber blend, superabsorbent fiber, superabsorbent powder-fiber blend, and mixtures thereof.

### Perforated Adherent Layers and Perforated Hydrophobic Layers

In some embodiments, the wound dressings described herein comprise a second layer, wherein the second layer is a perforated adherent layer. In some embodiments, the wound dressings described herein comprise a perforated adherent layer. In some embodiments, the perforated adherent layer comprises a perforated hydrophobic layer. In some embodiments, the perforated adherent layer is not hydrophobic, as may be the case when the perforated adherent layer comprises a hydrogel adhesive. In some embodiments, the wound dressings described herein comprise an adherent or hydrophobic layer selected from the group consisting of an adherent or hydrophobic layer comprising cuts, an adherent or hydrophobic layer comprising slits, an adherent or hydrophobic layer comprising holes, an adherent or hydrophobic layer comprising apertures, an adherent or hydrophobic layer comprising discontinuities, and an adherent or hydrophobic layer comprising bevels. In some embodiments, the perforated adherent layer or perforated hydrophobic layer is selected from the group consisting of silicone adhesive, hydrocolloid adhesive, polyurethane adhesive, rubber-based adhesive, acrylic adhesive, coated woven material, hydrogel adhesive, and combinations thereof. In some embodiments, the distribution and spacing of the perforations are regularly arranged with a separation substantially greater than their area. In some embodiments, the perforations are in a shape selected from a circle, a square, a rectangle, a triangle, an oval, a pentagon, a hexagon, and a rounded rectangle. In some embodiments, the perforations are circular and between 0.1 mm and 5 mm, or between 0.5 mm and 2 mm. In some embodiments, the spacing between the perforations is between 0.2 and 10 mm. In some embodiments, the number of perforations per unit area is between 1 and 100, or between 1 and 50, or between 1 and 20 perforations/cm². In some embodiments, the wound dressings disclosed herein comprise an open structure, hydrophobic layer comprising a material selected from the group consisting of silicone adhesive, hydrocolloid adhesive, polyurethane adhesive, hydrogel, acrylic adhesive, coated woven material, and mixtures thereof.

### Adhesives

In some embodiments, the wound dressings described herein comprise an adhesive surrounding the absorbent layer that adheres the dressing to the wound. In some embodiments, the absorbent layer is a non-adherent layer. The adhesive holds the absorbent component in direct contact with the wound and may seal the dressing to the skin surrounding the wound. The adhesive is preferably a silicone adhesive and more preferably a pressure sensitive silicone adhesive such as Dow Corning MD7-4502 or MG7-9900 or MG7-9800 (DowDuPont, USA) or Wacker Chemie AG SILPURAN® 2114, 2117, 2122, or 2142 (Wacker Chemie, Germany). The adhesive may also be a hydrocolloid, polyurethane, rubber based adhesive or acrylic adhesive.

### Foam Layers

In some embodiments, the wound dressings described herein comprise a foam layer. The foam layer can be an open cell foam layer. The foam layer can be a hydrophilic foam layer. In some embodiments, the hydrophilic foam layer is a polyurethane foam, such as a hydrophilic open celled foam. The foam typically has a thickness of 0.25 mm to 5 mm, preferably from 1 mm to 4.0 mm and most preferably from 1.5 mm to 3 mm. The foam layer preferably has an absorbency of 10 to 30 g/g, or 10 to 20 g/g when measured by the free swell absorptive capacity method. In some embodiments, the foam layer includes a metal-based antimicrobial agent that undergoes a controlled release when the binder layer comes into contact with moisture. In some embodiments, the foam layer includes an inorganic antimicrobial agent. In some embodiments, the foam layer does not include an inorganic antimicrobial agent.

The foam layer may be bonded to the wound contacting layer preferably by a polymer based melt layer, by an adhesive, by flame lamination, by ultrasound, or by curing directly to the foam layer. The foam layer may be directly bonded to the wound contact layer to make a laminate structure where the layers co-extend and are separated by the bonding line or the foam layer may form an island in the upper surface of the component surrounded by the wound contacting layer. By forming an island of foam in the upper surface of the absorbent component in this way, the tendency of the foam to laterally spread the exudate in the foam layer and rewet the wound contacting layer can be physically limited.

A textile layer may be positioned between the wound contact layer and the foam layer to limit distortion of the component that may occur when the foam layer expands on absorption of exudate. The textile layer is preferably made from absorbent fibers such as polyester, nylon, or cotton which may contain superabsorbent components such as cross linked sodium polyacrylate or may be made from a superabsorbent fiber such as polyacrylate.

In some embodiments, a one-way wicking layer is positioned between the wound contact layer and the foam layer to assist in the prevention of exudate rewetting the wound contact layer outside the area of the wound by transfer down from the foam towards the wound. The one-way wicking layer has the property that it resists the passage of exudate in one direction. The one-way wicking layer may be an embossed perforated film made from ethylene-methyl acrylate/ethylene vinyl acetate.

### Adaptable Wound Dressings

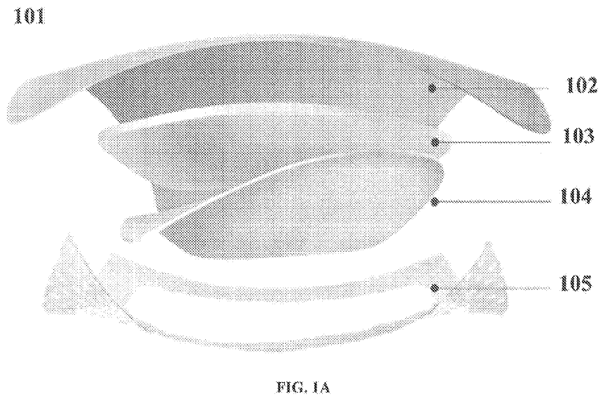

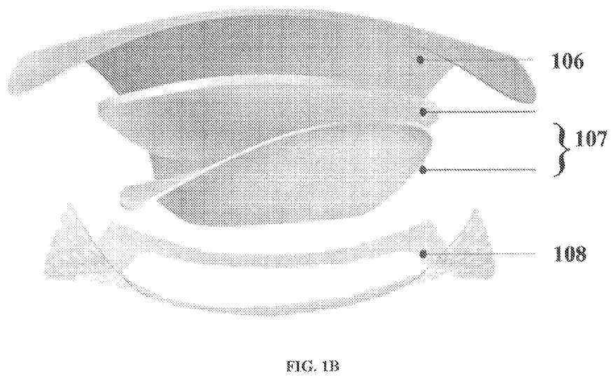

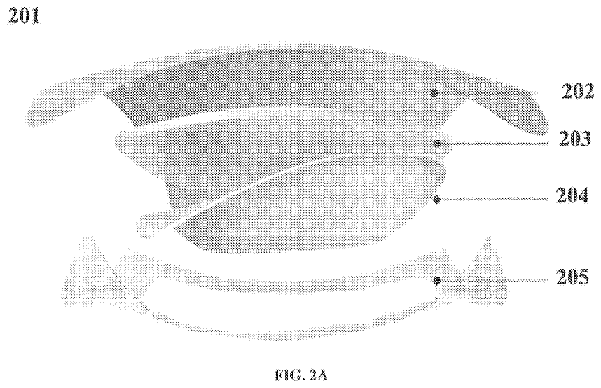

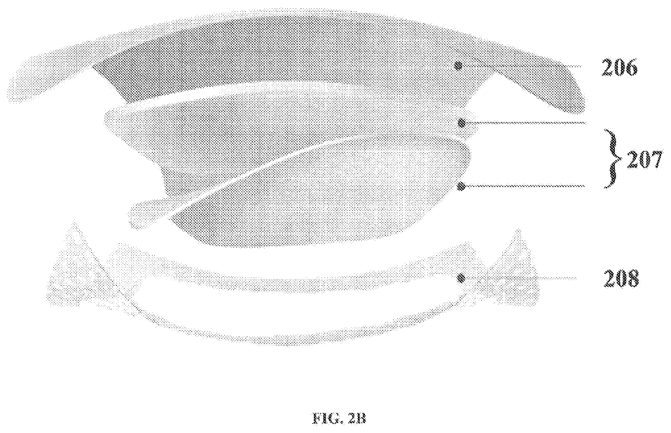

In some embodiments, provided herein are multilayered wound dressing that can be adapted at the point of use to provide either a wound contact layer comprising an absorbent material (including, for example, gelling fibers, absorbent fibers, or hydrophilic foam) or a wound contact layer comprising a perforated hydrophobic layer (including, for example, silicone adhesive, silicone gel, hydrocolloid adhesive, polyurethane adhesive, rubber-based adhesive, acrylic adhesive, or coated woven material). In some embodiments, the wound dressings are adaptable by either leaving intact a perforated hydrophobic wound contact layer, or by removing a part of the perforated hydrophobic wound contact layer in order to create a window in the perforated hydrophobic wound contact layer that exposes a hydrophilic wound contact layer, for example, a hydrophilic wound contact layer that comprises gelling fibers, absorbent fibers, non-adherent antimicrobial fibers, or is hydrophilic or substantially hydrophilic. In some embodiments, the window is made of silicone. In some embodiments, the silicon window has a non-woven or scrim layer adhered to the acrylic side to ensure that the window does not become stuck to a fibrous component. In some embodiments, the silicone window comprises at least one tab. In some embodiments, the silicone window is removed by pulling away at the at least one tab. In other embodiments, the silicone window is removed by pulling away at the at least one tab in a horizontal plane. In another embodiment, the tabs are suitably compatible with the silicone window when in place. A multilayered wound dressing (101) in accordance with aspects of the wound dressings disclosed herein is shown in FIG. 1A. The dressing may be in the form of a laminate including a polyurethane film layer (102), a polyurethane foam layer (103), a HYDROFIBER® (104), and a perforated silicone adhesive border (105). A multilayered wound dressing in accordance with aspects of the wound dressings disclosed herein is shown in FIG. 1B. The dressing may be in the form of a laminate including a cover layer (106), a first layer (107) which may comprise one or more layers, and a second layer (108) or wound contact layer which may have openings such as perforations, apertures, fenestrations, or the like and which provides a window exposing the first layer when at least a part of the release liner is removed. A multilayered wound dressing (201) in accordance with aspects of the wound dressings disclosed herein is shown in FIG. 2A. The dressing may be in the form of a laminate including a polyurethane film layer (202), a polyurethane foam layer (203), a HYDROFIBER® layer (204), and a perforated silicone adhesive border (205). A multilayered wound dressing in accordance with aspects of the wound dressings disclosed herein is shown in FIG. 2B. The dressing may be in the form of a laminate including a cover layer (206), a first layer (207) which may comprise one or more layers, and a second layer (208) or wound contact layer which may have openings such as perforations, apertures, fenestrations, or the like.

In some embodiments, the adaptable wound dressings disclosed herein are adaptable through the removal of a release liner that exposes a hydrophilic wound contact layer. In some embodiments, the adaptable wound dressings disclosed herein are adaptable through the removal of stitches or threads that expose a hydrophilic wound contact layer. In some embodiments, the adaptable wound dressings disclosed herein are adaptable through the removal of a bidirectional release liner that exposes a hydrophilic wound contact layer. In some embodiments, the adaptable wound dressings disclosed herein are adaptable through the removal of one or more tabs that exposes a hydrophilic wound contact layer. In some embodiments, the adaptable wound dressings disclosed herein are adaptable through the removal of indentations that exposes a hydrophilic wound contact layer. In other embodiments, the release liner comprises a printed indicator, indentation, raised bumps or other physical indication to allow a user to identify which contact layer will be exposed upon removing the liner in that direction.

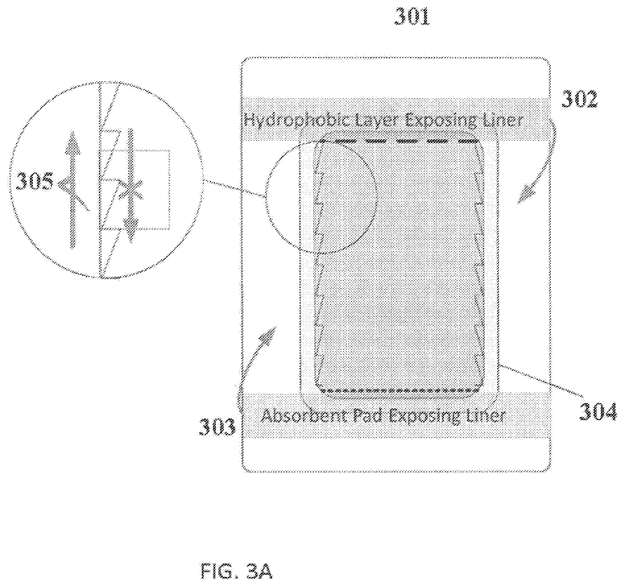

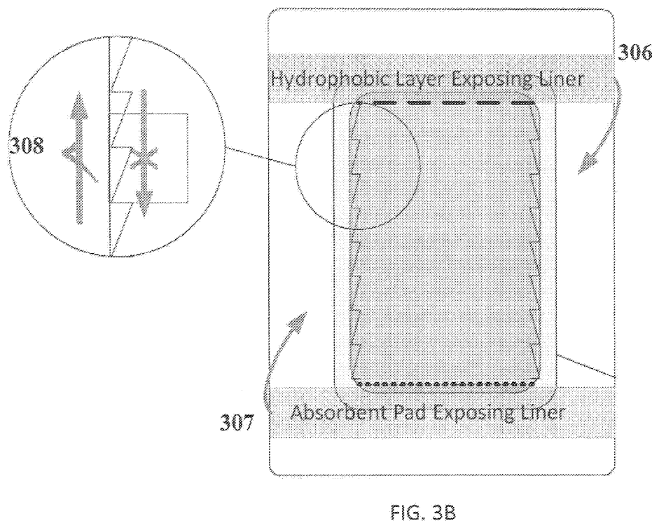

In some embodiments, the wound dressings disclosed herein comprise an upper backing film, an absorbent pad (comprising an absorbent hydrophilic wound facing layer such as a HYDROFIBER® layer; ConvaTec, United Kingdom), and a perforated open structure hydrophobic layer (such as a perforated silicone adhesive) protected by a release liner system. In some embodiments, the release liner system enables release of a portion of the open structure hydrophobic layer. In some embodiments, the release of a portion of the open structure hydrophobic layer is achieved via differential directional release (for example, removing the release layer by pulling from left to right exposes the perforated hydrophobic wound contact layer, whereas removing the release layer by pulling from right to left exposes the hydrophilic wound contact layer). In some embodiments, the differential directional release is enabled using zig-zag perforations, which allow exposure of the hydrophilic wound contact layer by “unzipping” the hydrophobic wound contact layer in one direction only. In some embodiments, the zig-zag perforations are arranged across a wound pad window in such a way that depending on the direction of the release liner removal, either (1) a silicone wound contact layer remains intact and is exposed, or (2) small perforations along the hydrophilic exposing liner will easily tear and “unzip” a window of the silicone wound contact layer and finally tear along large perforations on the opposite side of the wound dressing. Such an arrangement is depicted in FIG. 3, in which the small perforations are depicted as black dots running perpendicular to the wound dressing along the right-hand side of FIG. 3, and the large perforations are depicted as black dashes running perpendicular to the wound dressing along the left-hand side of FIG. 3. In the example depicted in FIG. 3, the release liner dictates which wound contact layer is exposed; there is only one central release liner and the direction of removal determines whether the silicone wound contact layer or the HYDROFIBER® wound contact is exposed (ConvaTec, United Kingdom). In some embodiments, the perforations and release liner system contribute to the functionality of the wound dressing. In some embodiments, the zig-zag perforations are arranged in such an orientation and geometry that when the zig-zag perforations are engaged the differential directional release of the release liner is achieved. As shown in FIG. 3A, an exemplary wound dressing (301) may contain an absorbent pad (304), and zig-zag perforations (305) allowing the hydrophobic layer to “unzip” in one direction only when the absorbent wound pad exposing liner is removed. Removal in the direction of arrow (303) (from right to left) exposes the absorbent pad wound contact layer. Removal in the direction of arrow (302) (from left to right) exposes the hydrophobic wound contact layer. As shown in FIG. 3B, an exemplary wound dressing may contain zig-zag perforations (308) allowing the hydrophobic layer to “unzip” in one direction only when the absorbent wound pad exposing liner is removed. Removal in the direction of arrow (307) (from right to left) exposes the absorbent pad wound contact layer. Removal in the direction of arrow (306) (from left to right) exposes the hydrophobic wound contact layer.

In some embodiments, the differential directional release is enabled using tabs located on opposite sides of the wound dressing. For example, in some embodiments, a wound dressing may contain two tabs: one tab located along a right-hand edge of the wound dressing that allows for exposure of a hydrophilic wound contact layer when the tab is pulled from right to left, and a second tab located along a left-hand edge of the wound dressing that allows for exposure of a perforated hydrophobic wound contact layer when the tab is pulled from left to right. In some embodiments, the differential directional release is enabled using adhesive technologies that provide directional release. In some embodiments, the differential directional release is enabled using a hook system. In some embodiments, the differential directional release is enabled using a loop and hook system. In some embodiments, the differential directional release is enabled without the use of adhesive.

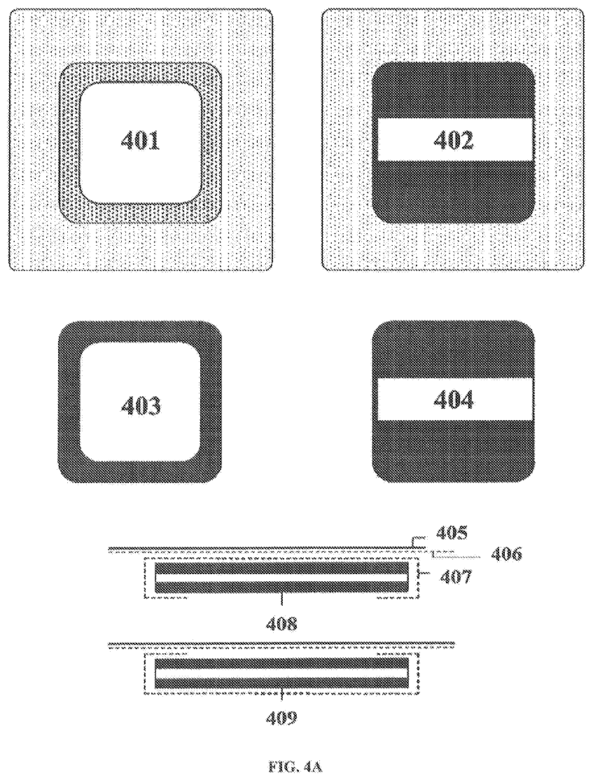

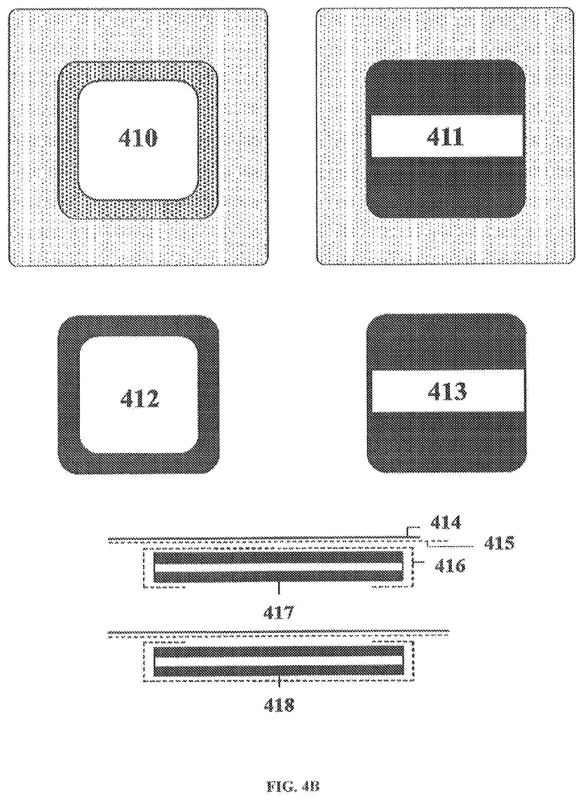

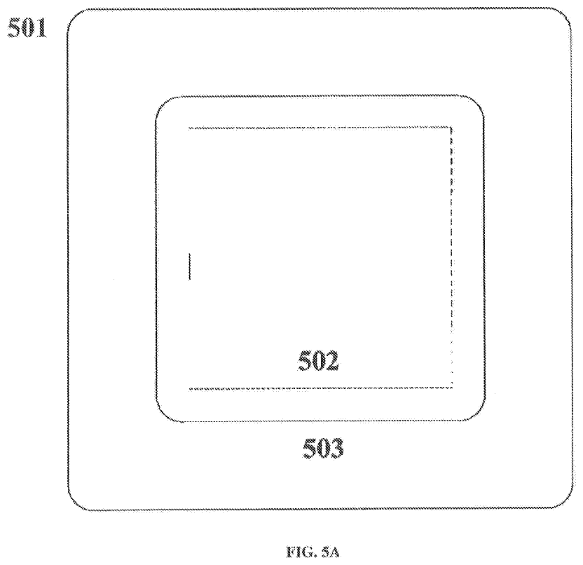

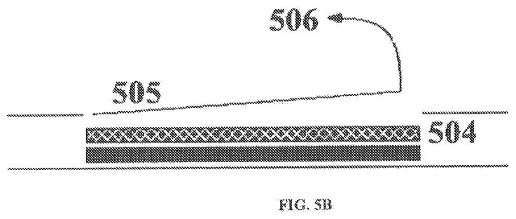

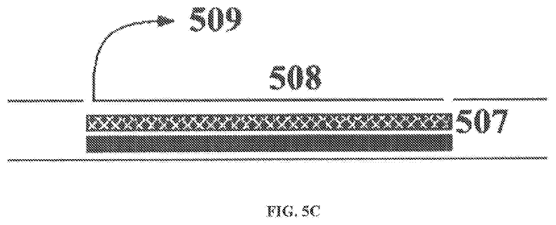

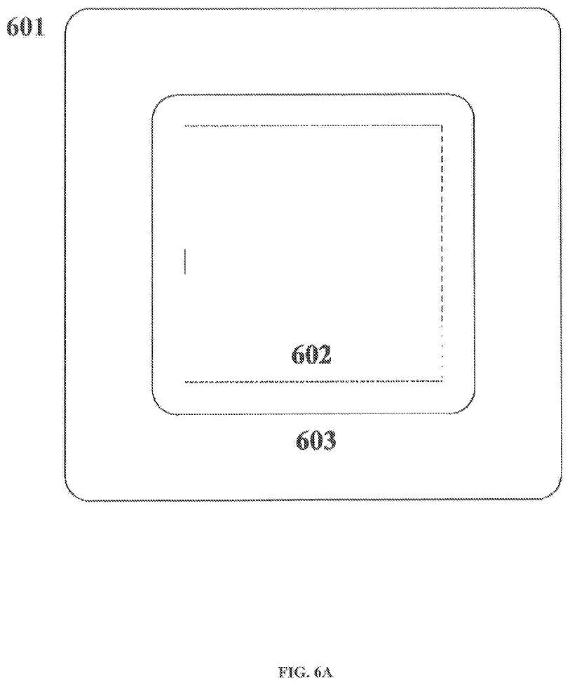

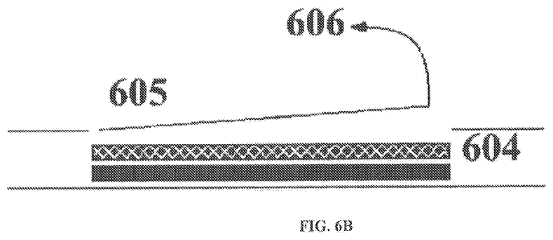

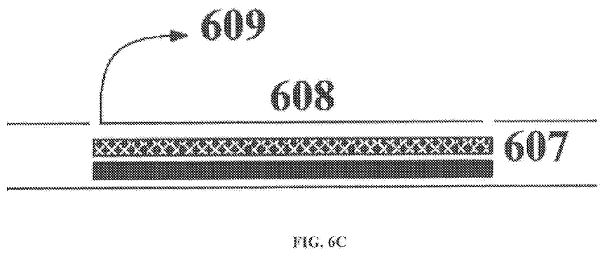

In some embodiments, the adaptable wound dressings disclosed herein are adaptable by reversing the side of the wound dressing that comes in contact with the wound. For example, in some embodiments, one side of a wound dressing comprises a hydrophilic absorbent wound interface and the opposite side of the wound dressing comprises an open structure hydrophobic/hydrophilic wound interface. In some embodiments, the adaptable wound dressings disclosed herein are adaptable via the use of a reversible absorbent pad. In some embodiments, the wound dressings disclosed herein comprise an absorbent pad comprising a layer of foam situated between two layers of carboxymethylcellulose encased in a layer of perforated silicone with a window cut out on one side to expose the carboxymethylcellulose and the other side comprising full perforated silicone with carboxymethylcellulose exposed through the perforations. In some embodiments, the wound dressings disclosed herein comprise an absorbent pad comprising a layer of foam situated between two layers of HYDROFIBER® (ConvaTec, United Kingdom) encased in a layer of perforated silicone with a window cut out on one side to expose the HYDROFIBER® (ConvaTec, United Kingdom) and the other side comprising full perforated silicone with HYDROFIBER® (ConvaTec, United Kingdom) exposed through the perforations. In some embodiments, the side of the reversible wound dressing with a window cut out is more suitable for medium- to high-exuding wounds. Medium- to high-exuding wounds may be characterized by soiled, wet, or heavy wound dressings that may leak or be malodorous. In some embodiments, medium- to high-exuding wounds may leak or be malodorous within 4, 6, 8, 10, 12, 14, 16, 18, 20, 22, or 24 hours. In some embodiments, medium- to high-exuding wounds may be characterized by soiled, wet, or heavy wound dressings within 4, 6, 8, 10, 12, 14, 16, 18, 20, 22, or 24 hours. In some embodiments, the side of the reversible wound dressing with full perforated silicone is more suitable for protection and for non- and for low-exuding wounds. In some embodiments, the reversible wound dressings disclosed herein comprise a backing film covered with a layer of perforated silicone on one side which enables either side of a reversible pad to be adhered to the backing film. Such an arrangement is depicted in FIG. 4A, in which the left-hand column depicts the HYDROFIBER® (ConvaTec, United Kingdom) exposed pad with a perforated silicone border (401 and 403). In FIG. 4A, element (405) is a backing film; element (406) is a perforated silicone adhesive layer; element (407) is a perforated silicone case surrounding an absorbent pad; and element (408) is an absorbent pad with a HYDROFIBER® (ConvaTec, United Kingdom) wound contact layer exposed (comprising an absorbent foam layer sandwiched in the middle between two HYDROFIBER® layers. The right-hand column of FIG. 4A depicts the perforated silicone wound contact layer with the HYDROFIBER® (ConvaTec, United Kingdom) layer obstructed by the perforated silicone wound contact layer except for the exposure at the perforations (402 and 404). In FIG. 4A, element (409) is an absorbent pad with a perforated silicone wound contact layer with partial HYDROFIBER® (ConvaTec, United Kingdom) exposure through the perforations. In some embodiments, the reversible wound dressings disclosed herein allow for removal of an absorbent pad from a backing film and exact placement of the absorbent pad onto a wound area, followed by covering of the pad with the backing film. In FIG. 4B, the left-hand column depicts the HYDROFIBER® (ConvaTec, United Kingdom) exposed pad with a perforated silicone border (410 and 412). In FIG. 4B, element (414) is a backing film; element (415) is a perforated silicone adhesive layer; element (416) is a perforated silicone case surrounding an absorbent pad; and element (417) is an absorbent pad with a HYDROFIBER® (ConvaTec, United Kingdom) wound contact layer exposed (comprising an absorbent foam layer sandwiched in the middle between two HYDROFIBER® layers. The right-hand column of FIG. 4B depicts the perforated silicone wound contact layer with the HYDROFIBER® (ConvaTec, United Kingdom) layer obstructed by the perforated silicone wound contact layer except for the exposure at the perforations (411 and 413). In FIG. 4B, element (418) is an absorbent pad with a perforated silicone wound contact layer with partial HYDROFIBER® (ConvaTec, United Kingdom) exposure through the perforations. In some embodiments, the reversible wound dressings disclosed herein allow for removal of an absorbent pad from a backing film and exact placement of the absorbent pad onto a wound area, followed by covering of the pad with the backing film. As shown in FIG. 5A, an exemplary wound dressing (501) in accordance with the wound dressings disclosed herein may contain an exposure window (502), an absorbent pad (503), and a series of discontinuities allowing for differential release of the hydrophobic layer or allowing it to remain in place. As shown in FIG. 5B, removal of the release liner (505) in the direction of arrow (506) (from right to left) allows the hydrophilic layer (504) to be exposed. As shown in FIG. 5C, removal of the release liner (508) in the direction of arrow (509) (from left to right) allows the hydrophobic layer (507) to remain intact. As shown in FIG. 6A, an exemplary wound dressing (601) in accordance with the wound dressings disclosed herein may contain an exposure window (602), an absorbent pad (603), and a series of discontinuities allowing for differential release of the hydrophobic layer or allowing it to remain in place. As shown in FIG. 6B, removal of the release liner (605) in the direction of arrow (606) (from right to left) allows the hydrophilic layer (604) to be exposed. As shown in FIG. 6C, removal of the release liner (608) in the direction of arrow (609) (from left to right) allows the hydrophobic layer (607) to remain intact.

### Methods of Manufacturing

In some embodiments, provided herein are methods of manufacturing adaptable adhesive wound dressings. In some embodiments, the methods of manufacturing adaptable adhesive wound dressings disclosed herein comprise forming a backing, forming an absorbent layer, forming a perforated hydrophobic layer, and forming a release liner comprising at least a first edge located opposite to a second edge, wherein the release liner is formed such that removal of the release liner by pulling from the first edge toward the second edge exposes the absorbent layer, and alternatively, removal of the release liner by pulling from the second edge toward the first edge exposes the perforated hydrophobic layer.

In some embodiments, the methods of manufacturing adaptable adhesive wound dressings disclosed herein comprise forming a backing, forming an adhesive layer, forming an absorbent layer, and forming a perforated hydrophobic layer encapsulating the absorbent layer on all but one side, wherein the perforated hydrophobic layer is formed such that either the first side or a second side which is opposite to the first side may be adhered to the adhesive layer.

In some embodiments, the wound dressings disclosed herein comprise one or more layers manufactured by a method selected from the group consisting of: needle punching, spunlacing, wet-laying, dry-laying, meltblowing, needle bonding, stitch-bonding, hydroentanglement, and felting. In some embodiments, the wound dressings disclosed herein comprise an absorbent layer manufactured by a method selected from the group consisting of: needle punching, spunlacing, wet-laying, dry-laying, meltblowing, needle bonding, stitch-bonding, hydroentanglement, and felting. In some embodiments, the absorbent layer is stitch bonded with strengthening fibers or yarns to provide additional strength to the absorbent layer such that it retains its structure when saturated with exudate. In some embodiments, the stitch-bonded structure affords higher absorbency or a degree of extensibility to the dressing depending on the nature of the strengthening fibers and yarns used and their stitch-bonding pattern. In some embodiments, the wound dressings disclosed herein comprise an absorbent layer comprising gel forming fibers bound to a foam layer formed by a method selected from the group consisting of: a polymer based melt layer, an adhesive, flame lamination and ultrasound. In some embodiments, the foam layer is directly bonded to the gel forming fibers to make a laminate structure where the layers co-extend and are separated by the bonding line. In some embodiments, the foam layer may form an island in the upper surface of the absorbent layer, physically limiting the tendency of the foam to laterally spread the exudate in the foam layer and rewet the absorbent layer.

In some embodiments, the methods of manufacturing adaptable adhesive wound dressings disclosed herein comprise needling together at least one member of the group selected from a backing, an absorbent layer, a perforated hydrophobic layer, and a release liner to another member of the group selected from a backing, an absorbent layer, a perforated hydrophobic layer, and a release liner. In some embodiments, the methods of manufacturing adaptable adhesive wound dressings disclosed herein comprise pressing together at least one member of the group selected from a backing, an absorbent layer, a perforated hydrophobic layer, and a release liner to another member of the group selected from a backing, an absorbent layer, a perforated hydrophobic layer, and a release liner.

In some embodiments, the methods of manufacturing adaptable adhesive wound dressings disclosed herein comprise needling together at least one member of the group selected from a backing, an adhesive layer, an absorbent layer, and a perforated hydrophobic layer to another member of the group selected from a backing, an adhesive layer, an absorbent layer, and a perforated hydrophobic layer. In some embodiments, the methods of manufacturing adaptable adhesive wound dressings disclosed herein comprise pressing together at least one member of the group selected from a backing, an adhesive layer, an absorbent layer, and a perforated hydrophobic layer to another member of the group selected from a backing, an adhesive layer, an absorbent layer, and a perforated hydrophobic layer.

In some embodiments, the methods of manufacturing adaptable adhesive wound dressings disclosed herein comprise applying a non-silicone side of a trilaminate wound dressing. In some embodiments, application of the non-silicone side of the trilaminate wound dressing can comprise application by flood coating, scatter coating, gradient coating, pattern coating, or grid lacquer. In some embodiments, the non-silicone side of the trilaminate wound dressings can be a thermal sensitive adhesive. In some embodiments, the non-silicone side of the trilaminate can be a “low tack” acrylic with a polycarbonate peel (such as from carboxymethylcell.ulose fiber) of less than 1.5 N/2.5 cm.

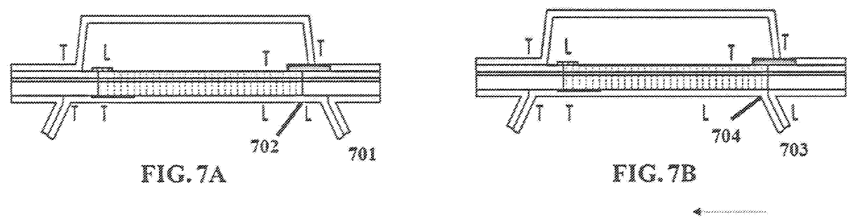

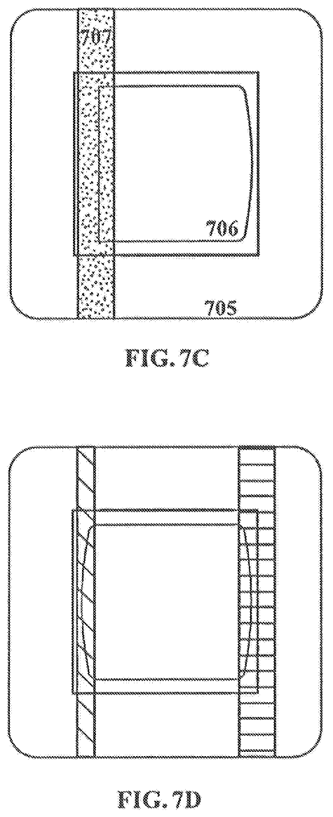

In some embodiments, the methods of manufacturing adaptable adhesive wound dressings disclosed herein comprises adhering the window to the release liner. In some embodiments, the positions of the release liner relative to the cuts in the window contribute to the removal or remaining of the window. In some embodiments, as depicted in FIG. 7A, the window has cuts (702) that are positioned inside the edges of the release liner (701). This arrangement increases the surface area of the window adhering to the release liner and creates a removal edge, where the peeling of release liner also peels the window to reveal the hydrophilic wound dressing underneath. In some embodiments, such as depicted in FIG. 7B, the edges of the release liner (703) are positioned immediately below the cuts (704) of the window. This arrangement decreases the surface area of the window adhering to the release liner and creates a stay edge, where peeling the release liner does not peel the window. In some embodiments, a light (L) or tight (T) release is positioned between the window and adjacent areas (FIG. 7A and FIG. 7B). In some embodiments, the L and T releases create the removal or stay edges of the multilayered wound dressing. In some embodiments, the L and T releases are used in conjunction with the relative positioning of the cuts of the window and the edges of the release liner to create the removal or stay edges of the multilayered wound dressing.

In some embodiments, the method of manufacturing adaptable adhesive wound dressings disclosed herein comprises additional adhesive (707) to the removal edge (FIG. 7C and FIG. 7D). In some embodiments, the additional adhesive creates tighter adhesion between the release liner (705) and the window (706). In some embodiments, the removal edge or the T release is created by laminating additional tape. In some embodiments, the tape is a polyester tape. In some embodiments, the removal edge of the T release is created by using more adhesive. In some embodiments, lighter adhesive is used to create the stay edge or the L release. In some embodiments, the lighter adhesive comprises using low-density polyethylene. In some embodiments, peeling release liner from the stay edge requires less force than peeling the release liner from the removal edge. In some embodiments, the T release and the L release are positioned between the releaser liner and the window or between the window and the hydrophilic wound dressing. In some embodiments, the edge with T release can be present or absent from the multilayered wound dressing. In some embodiments, the edge with T release conforms with the edge of the window.

### Methods of Treating Wounds

In some embodiment, provided herein are methods of treating wounds. One advantage of the adaptable wound dressings disclosed herein is that the user can choose which contact layer is best suited to the wound at the point of use. Use of the adaptable wound dressings disclosed herein for the treatment of wounds allows for the same type of adaptable wound dressing to be used throughout the healing process as the levels of exudate and the clinical needs vary. In some embodiments, the methods of treating a wound with the adaptable adhesive wound dressings described herein comprise determining a level of exudate from the wound. In some embodiments, the level of exudate from a wound is determined by weighing soiled wound dressings before and after application to a wound. In some embodiments, the level of exudate from a wound is determined by measuring the contents of wound drainage bags or canisters. In some embodiments, the level of exudate from a wound is determined based on the saturation of a wound dressing at specific intervals, such as every 24 or 48 hours. In some embodiments, the level of exudate from a wound is determined based on the appearance of the wound bed. For example, when a wound is highly exuding, the skin around the wound may be macerated and can be recognized by a whitish, plump, or soggy appearance which breaks down easily and can result in an increase in the overall size of the wound. Alternatively, for non to low-exuding wounds, the determination may be made by identifying that a wound dressing has dehydrated and caused a slight adhesion to the wound bed, in which case the dressing must be hydrated with fluid (such as saline) in order to allow removal of the wound dressing without discomfort to the patient.

In some embodiments, the methods of treating wounds disclosed herein comprise a determination that a wound is a medium- to high-exuding wound, followed by removal of a release liner from a wound dressing comprising a backing, an absorbent layer, and a perforated hydrophobic layer, such that removal of the release liner exposes the absorbent layer, which is then applied to the wound surface. In some embodiments, the methods of treating wounds disclosed herein comprise a determination that a wound is a non to low-exuding wound, followed by removal of a release liner from a wound dressing comprising a backing, an absorbent layer, and a perforated hydrophobic layer, such that removal of the release liner exposes the perforated hydrophobic layer, which is then applied to the wound surface.

In some embodiments, the methods of treating wounds disclosed herein comprise a determination that a wound is a medium- to high-exuding wound, followed by removal of an absorbent pad from a wound dressing comprising a backing, a perforated silicone adhesive layer, and an absorbent pad comprising a perforated silicone case surrounding an absorbent layer on all but one side, such that removal of the absorbent pad allows application of the one side without the surrounding perforated silicone onto the wound, followed by application of the backing onto the absorbent pad to cover the absorbent pad and adhere to the skin surrounding the wound. In some embodiments, the methods of treating wounds disclosed herein comprise a determination that a wound is a non to low-exuding wound, followed by removal of an absorbent pad from a wound dressing comprising a backing, a perforated silicone adhesive layer, and an absorbent pad comprising a perforated silicone case surrounding an absorbent layer on all but one side, such that removal of the absorbent pad allows application of a side with the surrounding perforated silicone onto the wound, followed by application of the backing onto the absorbent pad to cover the absorbent pad and adhere to the skin surrounding the wound.

### EXAMPLES

### List of Abbreviations

As used above, and throughout the description of the wound dressings described herein, the following abbreviations, unless otherwise indicated, shall be understood to have the following meanings:

| | |
|---|---|
| PU | polyurethane |
| CMC | carboxymethylcellulose |
| MVTR | moisture vapor transmission rate |

### Example 1: Treatment of a High-Exuding Wound

A medical care provider goes to check on the wound dressings covering a patient's wound. The medical care provider determines that the dressings are highly saturated with exudate, a fluid produced as part of the normal wound-healing process. The medical care provider identifies that the wound dressing was changed 24 hours ago and concludes that the patient has a high-exuding wound and that the wound dressing must be changed. The medical care provider obtains an adaptable adhesive wound dressing. See, e.g., FIG. 5A. The medical care provider removes the adaptable adhesive wound dressing from a protective wrapping and identifies that the adaptable adhesive wound dressing is a multilayered wound dressing with layers (in order from top to bottom) of: a polyurethane film backing; an absorbent layer comprising polyurethane foam and a hydrophilic layer comprising carboxymethylcellulose (CMC); a perforated hydrophobic layer comprising silicone; and a release liner. The adaptable adhesive wound dressing is printed with two opposing arrows: one arrow indicating the direction to pull the release liner for application to a medium- to high-exuding wound, and the other arrow indicating the direction to pull the release liner for application to a low- to non-exuding wound. The medical care provider removes the release liner from the adaptable adhesive wound dressing by pulling in the direction indicated on the adaptable adhesive wound dressing for application to a medium- to high-exuding wound. Pulling the release liner in this direction causes the release liner to tear away part of the perforated hydrophobic layer so that a window is tom into a perforated hydrophobic layer such that a hydrophilic layer comprising carboxymethylcellulose (CMC) is exposed. See FIG. 5B, FIG. 7A, and FIG. 7B. The medical care provider discards the release liner bound to part of the perforated hydrophobic layer and then applies the exposed hydrophilic layer or non-adherent layer comprising CMC to cover and protect the patient's wound whilst absorbing the level of exudate from the wound.

### Example 2: Treatment of a Low-Exuding Wound

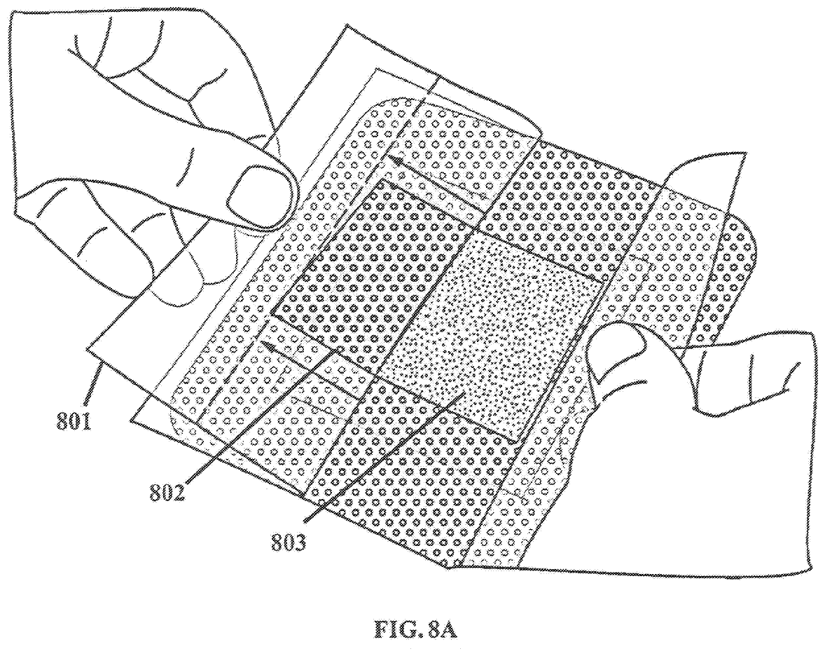

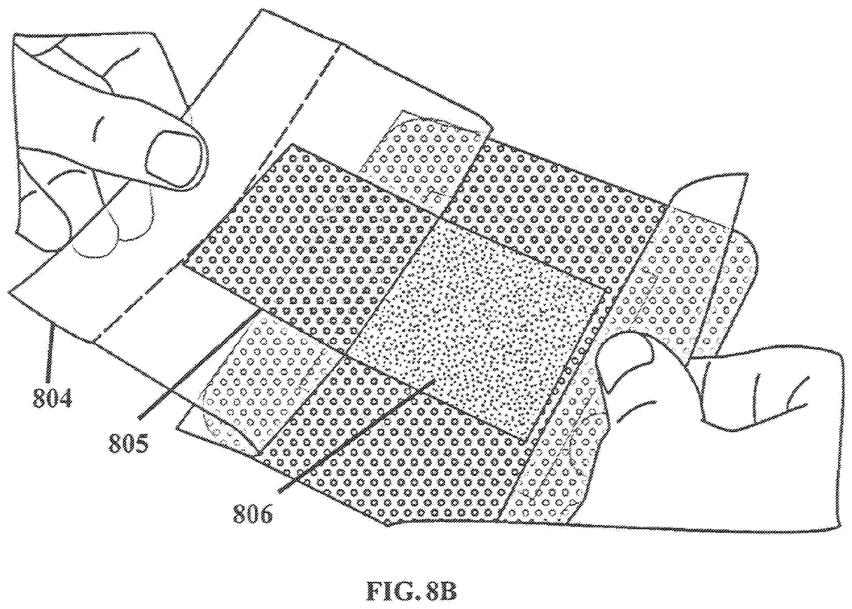

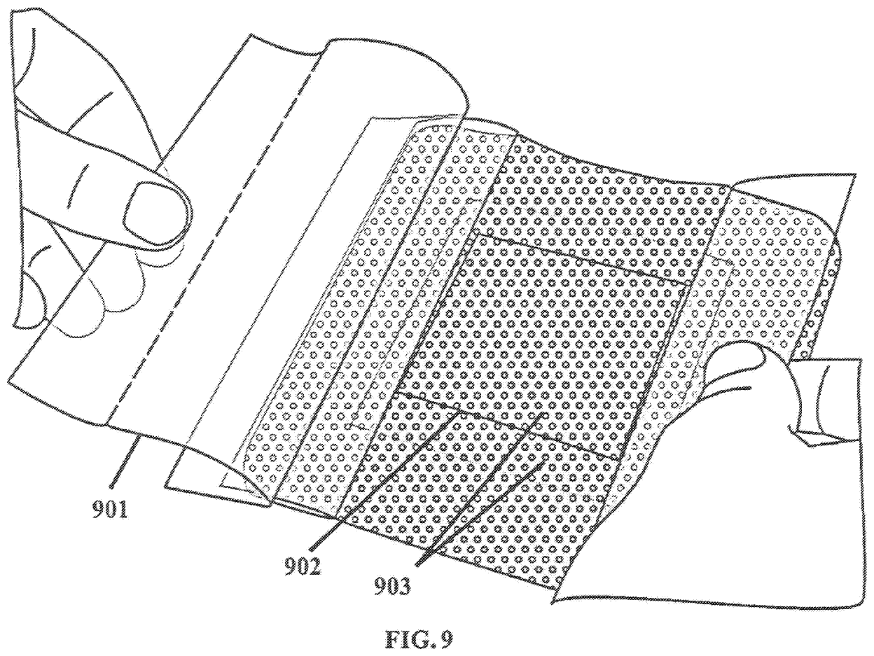

A medical care provider at a mobile clinic is presented with a patient with superficial wounds. The medical care provider determines that the patient's wound is a low-exuding wound, and requires a wound dressing that has minimal absorbency properties. The medical care provider obtains an adaptable adhesive wound dressing. See, e.g., FIG. 5A. The medical care provider removes a release liner from the adaptable adhesive wound dressing so that a perforated hydrophobic layer comprising polyurethane (PU) is exposed, removing the hydrophilic layer from the adaptive wound dressing. See FIG. 5C and FIG. 8. The medical care provider then applies the exposed hydrophobic layer comprising PU to cover and protect the patient's wound whilst absorbing the level of exudate from the wound. As depicted in FIG. 8A, removal of a release liner (801) peels back a window (802) through which a wound can be covered by a hydrophilic absorbent wound interface (803). As depicted in FIG. 8B, as the release liner (804) is fully peeled away, the removable window (805) further exposes the hydrophilic absorbent wound interface (806). As depicted in FIG. 9, the release liner (901) may be fully peeled away but leave an intact window (902) allowing a hydrophilic layer comprising both window and non-window portions (903) to remain.

In some embodiments, disclosed herein is a wound dressing comprising: a backing; an absorbent layer; a perforated hydrophobic layer; and a release liner comprising at least a first edge located opposite to a second edge, wherein said release liner is configured such that removal of said release liner from said first edge to said second edge exposes said absorbent layer, and removal of said release liner from said second edge to said first edge exposes said perforated hydrophobic layer.

In some embodiments, disclosed herein is a wound dressing wherein said release liner is configured such that removal of said release liner from said first edge to said second edge exposes said absorbent layer by dissociating said perforated hydrophobic layer from said wound dressing and said perforated hydrophobic layer remains associated with said release liner.

In some embodiments, disclosed herein is a wound dressing wherein dissociating said perforated hydrophobic layer comprises creating a window into said perforated hydrophobic layer through which said absorbent layer may contact a wound.

In some embodiments, disclosed herein is a wound dressing wherein said release liner is configured such that removal of said release liner from said second edge to said first edge exposes said perforated hydrophobic layer by dissociating said release liner from said wound dressing and said perforated hydrophobic layer remains associated with said wound dressing.

In some embodiments, disclosed herein is a wound dressing wherein said absorbent layer is selected from the group consisting of gelling fiber, absorbent fiber, hydrophilic foam, antimicrobial fibers, superabsorbent powder-fiber blends, non-gelling fibers, and combinations thereof.

In some embodiments, disclosed herein is a wound dressing wherein said absorbent layer comprises gel forming fibers bound to a foam layer.

In some embodiments, disclosed herein is a wound dressing wherein said foam layer comprises hydrophilic foam.

In some embodiments, disclosed herein is a wound dressing wherein said absorbent layer comprises a material selected from the group consisting of chemically modified cellulose, alginate, superabsorbent fibers, synthetic fibers, and combinations thereof.

In some embodiments, disclosed herein is a wound dressing wherein said chemically modified cellulose is carboxymethylcellulose or sodium carboxymethylcellulose.

In some embodiments, disclosed herein is a wound dressing wherein said backing further comprising an adhesive layer overlying said absorbent layer and extending beyond a periphery of said absorbent layer so as to adhere said wound dressing to skin surrounding a wound.

In some embodiments, disclosed herein is a wound dressing wherein the adhesive layer is selected from the group consisting of silicone adhesive, hydrocolloid adhesive, polyurethane adhesive, rubber-based adhesive, acrylic adhesive and combinations thereof.

In some embodiments, disclosed herein is a wound dressing wherein said perforated hydrophobic layer is selected from the group consisting of silicone adhesive, hydrocolloid adhesive, polyurethane adhesive, rubber-based adhesive, acrylic adhesive, coated woven material, non-woven material, knitted material, electrospun material, and combinations thereof.

In some embodiments, disclosed herein is a wound dressing wherein said backing comprises polyurethane film.

In some embodiments, disclosed herein is a wound dressing wherein said backing comprises polyurethane film laminated to polyurethane foam.

In some embodiments, disclosed herein is a wound dressing comprising: a backing; an adhesive layer; an absorbent layer; and a perforated hydrophobic layer encapsulating said absorbent layer on all but a first side, wherein said perforated hydrophobic layer is configured such that either said first side or a second side opposite to said first side may be adhered to said adhesive layer.

In some embodiments, disclosed herein is a wound dressing wherein said perforated hydrophobic layer encapsulates said absorbent layer creating a window through which said absorbent layer may contact a wound.

In some embodiments, disclosed herein is a wound dressing wherein said absorbent layer is selected from the group consisting of gelling fiber, non-gelling fiber, absorbent fiber, and hydrophilic foam, or combinations thereof.

In some embodiments, disclosed herein is a wound dressing wherein said absorbent layer comprises gel forming fibers bound to a foam layer.

In some embodiments, disclosed herein is a wound dressing wherein said foam layer comprises hydrophilic foam.

In some embodiments, disclosed herein is a wound dressing wherein said absorbent layer comprises a material selected from the group consisting of chemically modified cellulose, alginate, superabsorbent fibers, synthetic fibers, and combinations thereof.

In some embodiments, disclosed herein is a wound dressing wherein said chemically modified cellulose is carboxymethylcellulose or sodium carboxymethylcellulose.

In some embodiments, disclosed herein is a wound dressing wherein said adhesive layer comprises a layer of foam situated between two layers of sodium carboxymethylcellulose.

In some embodiments, disclosed herein is a wound dressing wherein the adhesive layer is selected from the group consisting of silicone adhesive, hydrocolloid adhesive, polyurethane adhesive, rubber-based adhesive, hydrogel, acrylic adhesive, and combinations thereof.

In some embodiments, disclosed herein is a wound dressing wherein said perforated hydrophobic layer is selected from the group consisting of silicone adhesive, hydrocolloid adhesive, polyurethane adhesive, rubber-based adhesive, acrylic adhesive, non-woven material, knitted material, and coated woven material.

In some embodiments, disclosed herein is a wound dressing wherein said backing comprises polyurethane film.

In some embodiments, disclosed herein is a wound dressing wherein said backing comprises polyurethane film laminated to polyurethane foam.

In some embodiments, disclosed herein is a method of manufacturing a wound dressing comprising: forming a backing; forming an absorbent layer; forming a perforated hydrophobic layer; and forming a release liner comprising at least a first edge located opposite to a second edge, wherein said release liner is formed such that removal of said release liner from said first edge to said second edge exposes said absorbent layer and removal of said release liner from said second edge to said first edge exposes said perforated hydrophobic layer.

In some embodiments, disclosed herein is a method of manufacturing a wound dressing comprising: forming a backing; forming an adhesive layer; forming an absorbent layer; and forming a perforated hydrophobic layer encapsulating said absorbent layer on all but a first side, wherein said perforated hydrophobic layer is formed such that either said first side or a second side opposite to said first side may be adhered to said adhesive layer.

In some embodiments, disclosed herein is a method of treating a wound comprising: determining a level of exudate from said wound; and, applying a wound dressing comprising: a backing; an absorbent layer; a perforated hydrophobic layer; and a release liner comprising at least a first edge located opposite to a second edge, configured such that removal of said release liner from said first edge to said second edge exposes said absorbent layer and removal of said release liner from said second edge to said first edge exposes said perforated hydrophobic layer whereupon a determination that said wound is a medium to high-exuding wound, the step of applying the wound dressing comprises removal of said release liner from said first edge to said second edge exposing the absorbent layer by removal of the perforated hydrophobic layer simultaneous with removal of the release liner and application of said wound dressing such that said absorbent layer is in contact with said wound.

In some embodiments, disclosed herein is a method of treating a wound comprising: determining a level of exudate from said wound; and, applying a wound dressing comprising: a backing; an absorbent layer; a perforated hydrophobic layer; and a release liner comprising at least a first edge located opposite to a second edge, configured such that removal of said release liner from said first edge to said second edge exposes said absorbent layer and removal of said release liner from said second edge to said first edge exposes said perforated hydrophobic layer whereupon a determination that said wound is a low- to non-exuding wound, the step of applying the wound dressing comprises removal of said release liner from said second edge to said first edge exposing the perforated hydrophobic layer and application of said wound dressing such that said wound dressing protects the wound.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A wound dressing comprising:
(a) a backing;
(b) an absorbent layer;
(c) a perforated hydrophobic layer; and
(d) a release liner comprising at least a first edge located opposite to a second edge,
wherein said release liner is configured such that removal of said release liner from said first edge to said second edge exposes said absorbent layer, and removal of said release liner from said second edge to said first edge exposes said perforated hydrophobic layer.

2. The wound dressing of claim 1, wherein said release liner is configured such that removal of said release liner from said first edge to said second edge exposes said absorbent layer by dissociating said perforated hydrophobic layer from said wound dressing and said perforated hydrophobic layer remains associated with said release liner.

3. The wound dressing of claim 2, wherein dissociating said perforated hydrophobic layer comprises creating a window into said perforated hydrophobic layer through which said absorbent layer may contact a wound.

4. The wound dressing of claim 1, wherein said release liner is configured such that removal of said release liner from said second edge to said first edge exposes said perforated hydrophobic layer by dissociating said release liner from said wound dressing and said perforated hydrophobic layer remains associated with said wound dressing.

5. The wound dressing of claim 1, wherein said absorbent layer is selected from the group consisting of gelling fiber, absorbent fiber, hydrophilic foam, antimicrobial fibers, superabsorbent powder-fiber blends, non-gelling fibers, and combinations thereof.

6. The wound dressing of claim 1, wherein said absorbent layer comprises gel forming fibers bound to a foam layer.

7. The wound dressing of claim 6, wherein said foam layer comprises hydrophilic foam.

8. The wound dressing of claim 1, wherein said absorbent layer comprises a material selected from the group consisting of chemically modified cellulose, alginate, superabsorbent fibers, synthetic fibers, and combinations thereof.

9. The wound dressing of claim 8, wherein said chemically modified cellulose is carboxymethylcellulose or sodium carboxymethylcellulose.

10. The wound dressing of claim 1, wherein said backing further comprising an adhesive layer overlying said absorbent layer and extending beyond a periphery of said absorbent layer so as to adhere said wound dressing to skin surrounding a wound.

11. The wound dressing of claim 10, wherein the adhesive layer is selected from the group consisting of silicone adhesive, hydrocolloid adhesive, polyurethane adhesive, rubber-based adhesive, hydrogel, acrylic adhesive and combinations thereof.

12. The wound dressing of claim 1, wherein said perforated hydrophobic layer is selected from the group consisting of silicone adhesive, hydrocolloid adhesive, polyurethane adhesive, rubber-based adhesive, acrylic adhesive, hydrogel, coated woven material, non-woven material, knitted material, electrospun material, and combinations thereof.

13. The wound dressing of claim 1, wherein said backing comprises polyurethane film.

14. The wound dressing of claim 1, wherein said backing comprises polyurethane film laminated to polyurethane foam.

15. A wound dressing comprising:
(a) a backing;
(b) an adhesive layer;
(c) an absorbent layer; and
(d) a perforated hydrophobic layer encapsulating said absorbent layer on all but a first side,
wherein said perforated hydrophobic layer is configured such that either said first side or a second side opposite to said first side may be adhered to said adhesive layer.

16. The wound dressing of claim 15, wherein said perforated hydrophobic layer encapsulates said absorbent layer creating a window through which said absorbent layer may contact a wound.

17. The wound dressing of claim 15, wherein said absorbent layer is selected from the group consisting of gelling fiber, non-gelling fiber, absorbent fiber, and hydrophilic foam, or combinations thereof.

18. The wound dressing of claim 15, wherein said absorbent layer comprises gel forming fibers bound to a foam layer.

19. The wound dressing of claim 18, wherein said foam layer comprises hydrophilic foam.

20. The wound dressing of claim 15, wherein said absorbent layer comprises a material selected from the group consisting of chemically modified cellulose, alginate, superabsorbent fibers, synthetic fibers, and combinations thereof.

21. The wound dressing of claim 20, wherein said chemically modified cellulose is carboxymethylcellulose or sodium carboxymethylcellulose.

22. The wound dressing of claim 15, wherein said adhesive layer comprises a layer of foam situated between two layers of sodium carboxymethylcellulose.

23. The wound dressing of claim 15, wherein the adhesive layer is selected from the group consisting of silicone adhesive, hydrocolloid adhesive, polyurethane adhesive, rubber-based adhesive, hydrogel, acrylic adhesive, and combinations thereof.

24. The wound dressing of claim 15, wherein said perforated hydrophobic layer is selected from the group consisting of silicone adhesive, hydrocolloid adhesive, polyurethane adhesive, rubber-based adhesive, acrylic adhesive, non-woven material, knitted material, and coated woven material.

25. The wound dressing of claim 15, wherein said backing comprises polyurethane film.

26. The wound dressing of claim 15, wherein said backing comprises polyurethane film laminated to polyurethane foam.

27-31. (canceled)
